(19) United States
(12) Patent Application Publication
(10) Pub. No.: US 2015/0056176 A1
(43) Pub. Date: Feb. 26, 2015

(54) ANTI-INFLAMMATORY COMPOSITION

(71) Applicants: STANLEY N. JANKOWITZ, Franklin Lakes, NJ (US); Raj K. Chopra, Westbury, NY (US)

(72) Inventors: STANLEY N. JANKOWITZ, Franklin Lakes, NJ (US); Raj K. Chopra, Westbury, NY (US)

(21) Appl. No.: 14/534,074

(22) Filed: Nov. 5, 2014

Related U.S. Application Data
(63) Continuation-in-part of application No. 14/215,870, filed on Mar. 17, 2014.
Application No. 14/215,870 is a continuation-in-part of application No. 13/353,169, filed on Jan. 18, 2012.

(51) Int. Cl. A61K 31/355; A61K 31/353; A61K 45/06; A61K 31/122

(52) U.S. Cl. CPC A61K 31/355; A61K 31/122; A61K 31/353; A61K 45/06
USPC 424/94.1; 514/690

(57) ABSTRACT

Disclosed herein are methods of using nutritional supplements and specific combinations thereof to counteract exercise-induced inflammation in monogastric organisms. Particular embodiments focus on use of quercetin, curcumin, boswellia, CoQ10, ribose, calcium carbonate, and zinc and their variants. Methods include ascertaining and modifying the nutritional supplements, ascertaining and comparing health states of all performance athletes, wherein the comparisons are made before and after modifications of the supplements and/or implementation of exercise regimens.

### CROSS-REFERENCE TO RELATED APPLICATIONS

The present application is a continuation-in-part of U.S. patent application Ser. No. 14/215,870 entitled “Equine Supplement,” filed Mar. 17, 2014, which claims priority to U.S. Provisional Patent Application Ser. No. 61/802,624 entitled “Equine Supplement”, filed Mar. 16, 2013, and this application is a continuation-in-part of U.S. patent application Ser. No. 13/353,169 entitled “Equine Nutritional Supplement”, filed Jan. 18, 2012, which claims priority to U.S. Provisional Patent Application Ser. No. 61/433,742 entitled “Nutritional Supplement”, filed Jan. 18, 2011, the disclosures of which are incorporated herein by reference in their entireties.

### BACKGROUND

1. Field of the Invention

Embodiments of the present invention are generally related to anti-inflammatory compositions and methods for supporting the health and wellbeing of monogastric organisms and preventing and/or treating various physical conditions. More specifically, embodiments of the present invention relate to administering quercetin, curcumin, boswellia, Coenzyme Q10 (CoQ10), ribose, calcium carbonate, zinc, and/or other vitamins and minerals to monogastric organisms.

2. Description of Related Art

Inflammation is a biological response of vascular tissues to harmful stimuli. The inflammatory response is the body's natural response that occurs immediately following tissue damage. Inflammation is a localized immune system response elicited by injury or stress, which serves to initiate the body's healing process. Its main functions are to defend the body against harmful substances, dispose of dead or dying tissue, and to promote the renewal of normal tissue. The inflammatory reaction may be characterized by signs that may include pain, swelling, redness, heat, loss of function, or the like.

While inflammation is a necessary and inevitable component of exertional adaptation, the inflammatory response of monogastric organisms and mammals to vigorous exercise is associated with the mild symptoms of delayed-onset muscle soreness and debilitating injuries related to soft tissue, joint and bone damage. Too much inflammation can cause negative symptoms such as swelling or pain and is believed to be a factor in some diseases if it occurs frequently. Certain types of joint pain are known to be caused by inflammation in the joint tissue. Inflammation is the initial problem that occurs in the joints. If allowed to continue without taking preventative measures, chronic inflammation can lead to significant joint problems such as degenerative joint disease.

Horses have joint problems because they are asked to perform activities beyond what they were “designed” to do. Horse competitions and shows may put a considerable amount of stress on a horse's joints. When joints suffer trauma, agents from the joint are released that destroy tissue inside the joint, including articular cartilage, resulting in traumatic arthritis. Arthritis is a form of joint disorder that involves inflammation of one or more joints. In horses with arthritis or related autoimmune diseases, the immune system triggers an inflammatory response when there are no foreign invaders to fight off. In these diseases, the body's normally protective immune system causes damage to its own tissues. The body responds as if normal tissues are infected or somehow abnormal. Inflammation can be chronic, for example from sustained or permanent injury or from autoimmune diseases. Other inflammation related sports injuries may occur in humans, horses, or other animals. Some of these sports injuries may include back pain, bone injuries including fractures, hamstring injuries, head injuries, heel pain, joint inflammation, knee pain, knee ligament damage, shoulder pain, and/or the like.

The standard of care for inflammation, arthritis, and related autoimmune diseases includes administration of prescription drugs. This standard of care has remained generally unchanged for over twenty years. Horses with increased levels of inflammation, such as horses 20 years of age and older, are often treated with non-steroidal anti-inflammatory drugs (NSAIDs) such as flunixin meglumine and phenylbutazone, or the like, to relieve pain and swelling. However, NSAIDs can cause health problems and do not repair the muscle or joint function damaged by inflammation. While anti-inflammatory medications may temporarily relieve muscle soreness, they do not reduce the mediators of inflammation or reduce the biomarkers of inflammation. These drugs do nothing to stop the bone and joint destruction caused by arthritis, or the like. Prolonged use of anti-inflammatory medication may increase the risk of more severe injury and other adverse effects. A study by Dirikolu and colleagues raised concerns that excessive use of these powerful drugs in horses might contribute to race-related injuries. Further, NSAIDS work by inhibiting the production of prostaglandins (PG) via inactivation or modification of the cyclooxygenase (COX) enzyme. Unfortunately disruption of the COX pathway can result in an increase in tumor necrosis factor alpha levels.

Less than 60% of thoroughbred 2-year-olds in training go on to race. Only 48% will still be racing as 3-year-olds. In 2010, starts per horse stood at 6.1, down almost 25% from 1990. The average number of career starts is approximately 16, a reduction of 40%-60% over the past 30 years. Respiratory disorders of the equine athlete are one of the major causes of reduced athleticism. Inflammatory airway disease (IAD) affects between 20%-80% of racehorses and performance horses. Horses with IAD exhibit a cough and exhibit excessive mucus and inflammatory sell accumulations in the tracheobronchial secretions.

With every living cell, there is an inflammatory spark waiting to ignite. More equine are permanently disabled because of joint, muscle, and/or respiratory inflammatory diseases than all other disease processes combined. Inflammation is also a culprit in lameness, which affects a horse's ability to train and perform. Accordingly, there is a renewed attention with respect to inflammatory and performance issues in equine or other performance athletes, or the like.

As such, a need exists for non-pharmacologic approaches to control or ameliorate exercise-induced inflammation in monogastric organisms, such as humans, dogs, cats, horses, and the like.

### SUMMARY

In one embodiment of the present invention a composition is provided that may comprise one or more of quercetin, curcumin, boswellia, Coenzyme Q10 (CoQ10), ribose. In some embodiments, the composition may additionally comprise calcium carbonate, and zinc. In some embodiments, a composition may comprise calcium carbonate, zinc, and curcumin. An additional embodiment of the present invention provides a method for ascertaining and comparing results of using nutraceuticals and/or nutritional supplements to counteract exercise-induced oxidative stress and its sequelae in a horse, the method comprising: determining dietary supplement and/or nutraceutical amounts for one or more of quercetin, curcumin, boswellia, CoQ10, ribose, calcium carbonate, and zinc, wherein the dietary supplement amounts and/or nutraceutical amounts are based at least in part on one or more ascertained vital characteristics of the horse; administering to the horse a dietary supplement containing one or more of quercetin, curcumin, boswellia, CoQ10, ribose, calcium carbonate, and zinc in the determined dietary supplement amounts; and ascertaining, with an equine blood-analysis apparatus, or the like, a health state of the horse, the ascertained health state of the horse being indicative of exercise-induced oxidative stress and its sequelae in the horse.

Another method in accordance with embodiments of the present invention comprising: modifying amount supplemented based at least in part on an ascertained health state of the horse; administering to the horse a nutraceutical and/or dietary supplement containing one or more of quercetin, curcumin, boswellia, CoQ10, ribose, calcium carbonate, and zinc in the modified nutraceutical and/or dietary supplement amounts; ascertaining, with an equine blood-analysis apparatus, or the like, a health state of the horse, the ascertained health state of the horse being indicative of exercise-induced oxidative stress and its sequelae in the horse; and determining, with a computer, or the like, a result of a comparison between the ascertained health states, the determined result being indicative of using quercetin, curcumin, boswellia, CoQ10, ribose, calcium carbonate, and zinc, as dietary supplements to counteract exercise-induced oxidative stress and its sequelae in a horse.

### DETAILED DESCRIPTION

It is understood that the embodiments of the present invention are not limited to the particular methodologies, protocols and the like, described herein as they may vary. It is also to be understood the terminology used herein is used for the purpose of describing particular embodiments only and not intended to limit the scope of the present invention. Unless defined otherwise, all technical and scientific terms used herein have the same meaning as commonly understood by one of ordinary skilled in the art to which this invention belongs. While some embodiments disclosed herein pertain to horses, it is appreciated that these embodiments may also pertain to humans, other monogastric organisms, and/or mammals. In some embodiments, a particular mammal may be discussed as an example. In each of these instances, these examples are also meant to include all other mammals, or the like, but in the interest of brevity and clarity, a single example mammal, such as a horse, may be described instead of listing all mammals.

The many embodiments of the presently disclosed invention relate in part to using nutritional supplements to achieve greater health and athletic performance within mammals and monogastric organisms, such as humans, dogs, cats, horses, and/or the like. While some embodiments described herein refer to equine or racehorses, it is contemplated that supplements and compositions in accordance with embodiments of the present invention may be administered to, and may achieve similar results in, all monogastric organisms. In some embodiments of the present invention a supplement and/or nutraceutical may be used with and/or be applied to other animals and/or species. Supplements and/or nutraceuticals in accordance with embodiments of the present disclosure may be used to as a preventative, rehabilitative, and/or curative measure in treating all monogastric organisms, including all performance equine during training, after training, when stressed, under high exertion.

Inflammation markers are associated with the production of pro-inflammatory cytokines, small hormone-like proteins produced in response to external stimuli. Repeated bouts of high intensity exercise may cause an increase in harmful pro-inflammatory cytokine expression, or the like. Pro-inflammatory cytokines such as interleukin (IL)-1β, IL-6 and tumor necrosis factor-alpha (TNFα) are induced by vigorous exercise. The consequences associated with this increased expression of inflammatory mediators post-exercise can range from the mild symptoms of delayed-onset muscle soreness to debilitating problems related to soft tissue, joint and bone damage. Chronic inflammation can lead to tissue destruction and muscle injuries, bone injuries, tissue injuries, and/or the like. In general, equine and/or human athletes exhibit evidence of increased inflammation following exercise as characterized by increased expression of mRNA for TNFα, IL-1β, and IL-6 in peripheral blood mononuclear cells.

Given the negative impact of exercise-induced inflammation, there is widespread use of non-steroidal anti-inflammatory drugs (NSAIDS) in humans and racehorses. A recent study reported that the plasma concentrations of NSAIDS were highest in injured horses. While anti-inflammatory medication may relieve muscle soreness in human athletes, they do not affect recovery of muscle function or markers of inflammation and may impede the inflammatory response. Outside of concerns regarding the possibility that excessive use of NSAIDS may contribute to race-related injuries, long-term use of NSAIDs is associated with other adverse effects. Thus, there is growing interest in non-pharmacologic approaches to control or ameliorate exercise-induced inflammation.

Pro-inflammatory mediators help regulate the body's normal acute and chronic inflammation. Injuries ending racing careers are sometimes due to the cumulative effect of the inflammatory cascade. This inflammation causes mild to moderate joint, muscle, bone, and tissue damage. This occurs over time at a rate that exceeds the affected tissues' capacity to heal itself. Clearly, the racing industry needs a new approach to managing pre- and post-exercise inflammation. A recent study assessed the cytokine response to exercise in a group of young Thoroughbred racehorses during their initial training. By harnessing the unmitigated inflammatory effects that result from intense training, the study revealed a reduction in the number of race training related injuries. This was possible after a nutraceutical complex in accordance with the present disclosure was administered.

There is a need for non-pharmacologic approaches to control or ameliorate exercise induced inflammation. The racing industry, in particular, is in desperate need of a new approach, a “seismic shift” to help manage the short and long term health of young thoroughbreds in training. Nutraceuticals and/or compositions in accordance with embodiments of the present invention are adapted to improve a horse's metabolic condition through the reduction of systemic-inflammatory-mediators.

Compositions in accordance with the present disclosure may also comprise quercetin, curcumin, boswellia, Coenzyme Q10 (CoQ10), ribose, calcium carbonate, and/or zinc. A composition in accordance with the present disclosure may comprise elements that synergistically work together to solve the inflammation problems discussed herein.

Quercetin is a polyphenolic antioxidant flavonoid found in plants and plays an important role in the inhibition of enzymes involved in free radical production. Quercetin can be used for the prevention and elevation of conditions that are associated with oxidative stress and may improve athletic performance by improving endurance and VO2 max (maximal oxygen update or aerobic capacity) in equine. Quercetin, as an anti-inflammatory supplementation, may increase fitness and maximal oxygen consumption by elevating the number of intracellular mitochondria.

Quercetin may improve exercise performance may help maintain healthy cytokines and inflammatory responses at the cellular levels, or the like. Quercetin may generally increase brain and muscle mitochondrial biogenesis and exercise tolerance. Quercetin has anti-inflammatory and antioxidant properties and scavenges free radicals, thereby preventing free radical inflammatory damage to the body. In exemplary embodiments, the quercetin may be water soluble. Flavonoids (quercetin) and polyphenolic compounds, curcuminoids, boswellia, and/or CoQ10, may reduce cellular inflammation and may outperform NSAIDs, or the like, by being effective by the ingredients working synergistically at lower doses.

Vitamin E serves to maintain neurological health and normal neuromuscular function. Deficiencies in vitamin E may lead to abnormal neuromuscular function and/or neurological diseases or disorders. For example, some exemplary neurologic diseases that may affect horses due to a deficiency in vitamin E may include equine degenerative myeloencephalopathy (EDM) and equine motor neuron disease (EMND), to name a few. Many other neurological disorders may be linked to genetic or exercise-induced vitamin E deficiencies, and are contemplated by and within embodiments of the present invention. In some embodiments, compositions, supplements, and/or nutraceuticals in accordance with the present invention may be used to prevent and/or treat horses, humans, or other animals for conditions, including physical and/or neurological disorders, caused by deficiencies in vitamin E, which may be naturally occurring or may occur as a result of stress, exercise, and/or the like. In some embodiments, the supplements disclosed herein may also be administered to support the respiratory system of an equine, to support endothelial cell function. Race horses often participate in high intensity exercise, which may have detrimental effects on the health and performance of the horses. Repeated bouts of high intensity exercise will lead to severe stress resulting in the depletion of endogenous CoQ10, which is the first line of defense among lipophilic antioxidants. The next in line to be depleted would be vitamin E, or tocopherols (α and/or γ-tocopherols), due to continued excessive oxidative stress. In some embodiments vitamin E may be included in the amount of 13 g, in the range of approximately 250 IUs to approximately 300 IUs, or from approximately 3 g to 26 g.

Generally, CoQ10 may potentiate the antioxidant effects of vitamin E, and may regenerate vitamin E from its oxidized form. Accordingly, CoQ10 has a sparing effect on vitamin E levels, such that CoQ10 may protect and/or regenerate vitamin E. In horses not receiving any CoQ10-containing dietary supplements, there is a marked decrease in plasma CoQ10 and/or vitamin E concentrations when they are subjected to stress or exercise such as high intensity training, and this could possibly lead to impaired health and poor physical performance. The composition may comprise natural vitamin E and/or synthetic vitamin E. For example, a daily dose of 1200-1600 IU of RRR-alpha tocopheryl acetate (natural vitamin E) may be administered to horses. In exemplary embodiments, administering a daily dose of natural vitamin E may maintain adequate plasma vitamin E in exercising horses. In some embodiments the amounts of the supplements and/or nutraceuticals in accordance with the present invention that are administered for preventative purposes may be different than amounts administered. For example administering a nutraceutical to reestablish vitamin E levels and for treatment dosages may be higher than administering a nutraceutical to prevent deficiencies in vitamin E and the health conditions related to vitamin E deficiency. In some embodiments, horses of different ages and/or weights may be administered different amounts. In some embodiments, supplements and/or nutraceuticals in accordance with the present disclosure may help promote a healthy immune system, or the like.

Administration of a composition in accordance with embodiments of the present invention may lead to a marked increase in plasma CoQ10 and vitamin E concentrations, improvements in health and performance, and prevention and/or treatment of many physical and/or neurological disorders in humans, horses, cats, dogs, and other monogastric organisms, or the like. In some embodiments, compositions, supplements, and/or nutraceuticals in accordance with the present invention may be used to prevent and/or treat humans, horses, cats, dogs, and other monogastric organisms, or the like for conditions, including physical and/or neurological disorders, caused by deficiencies in vitamin E, which may be naturally occurring or may occur as a result of stress, exercise, and/or the like. Supplementation with CoQ10 and vitamin E combined with quercetin, curcumin, boswellia, Coenzyme Q10 (CoQ10), vitamin E, ribose, calcium carbonate, and/or zinc may work synergistically to treat, prevent, and/or cure disorders such as Alzheimer's disease, dementia, disorders of the spine, disorders of the brain, disorders of the vascular system, and/or disorders of the heart. After administration, the anti-inflammatory elements of a composition in accordance with exemplary embodiments of the present disclosure may comprise quercetin, curcumin, boswellia, and ribose and may work alone and synergistically as anti-inflammatories and alone and synergistically with CoQ10 and vitamin E to treat, prevent, slow the progression, and/or cure disorders such as Alzheimer's disease, dementia, disorders of the spine, disorders of the brain, disorders of the vascular system, and/or disorders of the heart.

Curcumin may be administered to treat, prevent, and/or cure various central nervous system disorders. Curcumin has neuroprotective action in Alzheimer's disease, tardive dyskinesia, major depression, epilepsy, and other related neurodegenerative and neuropsychiatric disorders. Curcumin is an inhibitor of reactive astrocyte expression and thus prevents cell death. Curcumin also modulates various neurotransmitter levels in the brain. Alzheimer's disease is a progressive neurodegenerative disorder. Increased oxidative stress has been shown to be a prominent and early feature in Alzheimer's disease. Anti-inflammatories such as boswellia may be administered to treat, prevent, and/or cure neurological diseases tied to increased oxidative stress such as Alzheimer's disease, or the like.

Some examples of neurological disorders that may be treated, prevented, and/or cured by administration of a composition in accordance with exemplary embodiments of the present invention may include: Acid Lipase Disease; Acid Maltase Deficiency; Acquired Epileptiform Aphasia; Acute Disseminated Encephalomyelitis; ADHD; Adie's Pupil; Adie's Syndrome; Adrenoleukodystrophy; Agenesis of the Corpus Callosum; Agnosia; Aicardi Syndrome; Aicardi-Goutieres Syndrome Disorder; AIDS—Neurological Complications; Alexander Disease; Alpers' Disease; Alternating Hemiplegia; Alzheimer's Disease; Amyotrophic Lateral Sclerosis (ALS); Anencephaly; Aneurysm; Angelman Syndrome; Angiomatosis; Anoxia; Antiphospholipid Syndrome; Aphasia; Apraxia; Arachnoid Cysts; Arachnoiditis; Arnold-Chiari Malformation; Arteriovenous Malformation; Asperger Syndrome; Ataxia; Ataxia Telangiectasia; Ataxias and Cerebellar or Spinocerebellar Degeneration; Atrial Fibrillation and Stroke; Attention Deficit-Hyperactivity Disorder; Autism; Autonomic Dysfunction; Back Pain; Barth Syndrome; Batten Disease; Becker's Myotonia; Behcet's Disease; Bell's Palsy; Benign Essential Blepharospasm; Benign Focal Amyotrophy; Benign Intracranial Hypertension; Bernhardt-Roth Syndrome; Binswanger's Disease; Blepharospasm; Bloch-Sulzberger Syndrome; Brachial Plexus Birth Injuries; Brachial Plexus Injuries; Bradbury-Eggleston Syndrome; Brain and Spinal Tumors; Brain Aneurysm; Brain Injury; Brown-Sequard Syndrome; Bulbospinal Muscular Atrophy; CADASIL; Canavan Disease; Carpal Tunnel Syndrome; Causalgia; Cavernomas; Cavernous Angioma; Cavernous Malformation; Central Cervical Cord Syndrome; Central Cord Syndrome; Central Pain Syndrome; Central Pontine Myelinolysis; Cephalic Disorders; Ceramidase Deficiency; Cerebellar Degeneration; Cerebellar Hypoplasia; Cerebral Aneurysms; Cerebral Arteriosclerosis; Cerebral Atrophy; Cerebral Beriberi; Cerebral Cavernous Malformation; Cerebral Gigantism; Cerebral Hypoxia; Cerebral Palsy; Cerebro-Oculo-Facio-Skeletal Syndrome (COFS); Charcot-Marie-Tooth Disease; Chiari Malformation; Cholesterol Ester Storage Disease; Chorea; Choreoacanthocytosis; Chronic Inflammatory Demyelinating Polyneuropathy (CIDP); Chronic Orthostatic Intolerance; Chronic Pain; Cockayne Syndrome Type II; Coffin Lowry Syndrome; Colpocephaly; Coma; Complex Regional Pain Syndrome; Congenital Facial Diplegia; Congenital Myasthenia; Congenital Myopathy; Congenital Vascular Cavernous Malformations; Corticobasal Degeneration; Cranial Arteritis; Craniosynostosis; Cree encephalitis; Creutzfeldt-Jakob Disease; Cumulative Trauma Disorders; Cushing's Syndrome; Cytomegalic Inclusion Body Disease; Cytomegalovirus Infection; Dancing Eyes-Dancing Feet Syndrome; Dandy-Walker Syndrome; Dawson Disease; De Morsier's Syndrome; Deep Brain Stimulation for Parkinson's Disease; Dejerine-Klumpke Palsy; Dementia; Dementia—Multi-Infarct; Dementia—Semantic; Dementia—Subcortical; Dementia With Lewy Bodies; Dentate Cerebellar Ataxia; Dentatorubral Atrophy; Dermatomyositis; Developmental Dyspraxia; Devic's Syndrome; Diabetic Neuropathy; Diffuse Sclerosis; Dravet Syndrome; Dysautonomia; Dysgraphia; Dyslexia; Dysphagia; Dyspraxia; Dyssynergia Cerebellaris Myoclonica; Dyssynergia Cerebellaris Progressiva; Dystonias; Early Infantile Epileptic Encephalopathy; Empty Sella Syndrome; Encephalitis; Encephalitis Lethargica; Encephaloceles; Encephalopathy; Encephalopathy (familial infantile); Encephalotrigeminal Angiomatosis; Epilepsy; Epileptic Hemiplegia; Erb-Duchenne and Dejerine-Klumpke Palsies; Erb's Palsy; Essential Tremor; Extrapontine Myelinolysis; Fabry Disease; Fahr's Syndrome; Fainting; Familial Dysautonomia; Familial Hemangioma; Familial Idiopathic Basal Ganglia Calcification; Familial Periodic Paralyses; Familial Spastic Paralysis; Farber's Disease; Febrile Seizures; Fibromuscular Dysplasia; Fisher Syndrome; Floppy Infant Syndrome; Foot Drop; Friedreich's Ataxia; Frontotemporal Dementia; Gaucher Disease; Generalized Gangliosidoses; Gerstmann's Syndrome; Gerstmann-Straussler-Scheinker Disease; Giant Axonal Neuropathy; Giant Cell Arteritis; Giant Cell Inclusion Disease; Globoid Cell Leukodystrophy; Glossopharyngeal Neuralgia; Glycogen Storage Disease; Guillain-Barré Syndrome; Hallervorden-Spatz Disease; Head Injury; Headache; Hemicrania Continua; Hemifacial Spasm; Hemiplegia Alterans; Hereditary Neuropathies; Hereditary Spastic Paraplegia; Heredopathia Atactica Polyneuritiformis; Herpes Zoster; Herpes Zoster Oticus; Hirayama Syndrome; Holmes-Adie syndrome, Holoprosencephaly; HTLV-1 Associated Myelopathy; Hughes Syndrome; Huntington's Disease; Hydranencephaly; Hydrocephalus; Hydrocephalus—Normal Pressure; Hydromyelia; Hypercortisolism; Hypersomnia; Hypertonia; Hypotonia; Hypoxia; Immune-Mediated Encephalomyelitis; Inclusion Body Myositis; Incontinentia Pigmenti; Infantile Hypotonia; Infantile Neuroaxonal Dystrophy; Infantile Phytanic Acid Storage Disease; Infantile Refsum Disease; Infantile Spasms; Inflammatory Myopathies; Iniencephaly; Intestinal Lipodystrophy; Intracranial Cysts; Intracranial Hypertension; Isaacs' Syndrome; Joubert Syndrome; Kearns-Sayre Syndrome; Kennedy's Disease; Kinsbourne syndrome; Kleine-Levin Syndrome; Klippel-Feil Syndrome; Klippel-Trenaunay Syndrome (KTS); Klüver-Bucy Syndrome; Korsakoff's Amnesic Syndrome; Krabbe Disease; Kugelberg-Welander Disease; Kuru; Lambert-Eaton Myasthenic Syndrome; Landau-Kleffner Syndrome; Lateral Femoral Cutaneous Nerve Entrapment; Lateral Medullary Syndrome; Learning Disabilities; Leigh's Disease; Lennox-Gastaut Syndrome; Lesch-Nyhan Syndrome; Leukodystrophy; Levine-Critchley Syndrome; Lewy Body Dementia; Lipid Storage Diseases; Lipoid Proteinosis; Lissencephaly; Locked-In Syndrome; Lou Gehrig's Disease; Lupus—Neurological Sequelae; Lyme Disease—Neurological Complications; Machado-Joseph Disease; Macrencephaly; Megalencephaly; Melkersson-Rosenthal Syndrome; Meningitis; Meningitis and Encephalitis; Menkes Disease; Meralgia Paresthetica; Metachromatic Leukodystrophy; Microcephaly; Migraine; Miller Fisher Syndrome; Mini Stroke; Mitochondrial Myopathies; Moebius Syndrome; Monomelic Amyotrophy; Motor Neuron Diseases; Moyamoya Disease; Mucolipidoses; Mucopolysaccharidoses; Multifocal Motor Neuropathy; Multi-Infarct Dementia; Multiple Sclerosis; Multiple System Atrophy; Multiple System Atrophy with Orthostatic Hypotension; Muscular Dystrophy; Myasthenia—Congenital; Myasthenia Gravis; Myelinoclastic Diffuse Sclerosis; Myoclonic Encephalopathy of Infants; Myoclonus; Myopathy; Myopathy—Congenital; Myopathy—Thyrotoxic; Myotonia; Myotonia Congenita; Narcolepsy; Neuroacanthocytosis; Neurodegeneration with Brain Iron Accumulation; Neurofibromatosis; Neuroleptic Malignant Syndrome; Neurological Complications of AIDS; Neurological Complications of Lyme Disease; Neurological Consequences of Cytomegalovirus Infection; Neurological Manifestations of Pompe Disease; Neurological Sequelae Of Lupus; Neuromyelitis Optica; Neuromyotonia; Neuronal Ceroid Lipofuscinosis; Neuronal Migration Disorders; Neuropathy—Hereditary; Neurosarcoidosis; Neurosyphilis; Neurotoxicity; Nevus Cavernosus; Niemann-Pick Disease; Normal Pressure Hydrocephalus; Occipital Neuralgia; Ohtahara Syndrome; Olivopontocerebellar Atrophy; Opsoclonus Myoclonus; Orthostatic Hypotension; O'Sullivan-McLeod Syndrome; Overuse Syndrome; Pain—Chronic; Pantothenate Kinase-Associated Neurodegeneration; Paraneoplastic Syndromes; Paresthesia; Parkinson's Disease; Paroxysmal Choreoathetosis; Paroxysmal Hemicrania; Parry-Romberg; Pelizaeus-Merzbacher Disease; Pena Shokeir II Syndrome; Perineural Cysts; Periodic Paralyses; Peripheral Neuropathy; Periventricular Leukomalacia; Persistent Vegetative State; Pervasive Developmental Disorders; Phytanic Acid Storage Disease; Pick's Disease; Pinched Nerve; Piriformis Syndrome; Pituitary Tumors; Polymyositis; Pompe Disease; Porencephaly; Postherpetic Neuralgia; Postinfectious Encephalomyelitis; Post-Polio Syndrome; Postural Hypotension; Postural Orthostatic Tachycardia Syndrome; Postural Tachycardia Syndrome; Primary Dentatum Atrophy; Primary Lateral Sclerosis; Primary Progressive Aphasia; Prion Diseases; Progressive Hemifacial Atrophy; Progressive Locomotor Ataxia; Progressive Multifocal Leukoencephalopathy; Progressive Sclerosing Poliodystrophy; Progressive Supranuclear Palsy; Prosopagnosia; Pseudo-Torch syndrome; Pseudotoxoplasmosis syndrome; Pseudotumor Cerebri; Psychogenic Movement; Ramsay Hunt Syndrome I; Ramsay Hunt Syndrome II; Rasmussen's Encephalitis; Reflex Sympathetic Dystrophy Syndrome; Refsum Disease; Refsum Disease—Infantile; Repetitive Motion Disorders; Repetitive Stress Injuries; Restless Legs Syndrome; Retrovirus-Associated Myelopathy; Rett Syndrome; Reye's Syndrome; Rheumatic Encephalitis; Riley-Day Syndrome; Sacral Nerve Root Cysts; Saint Vitus Dance; Salivary Gland Disease; Sandhoff Disease; Schilder's Disease; Schizencephaly; Seitelberger Disease; Seizure Disorder; Semantic Dementia; Septo-Optic Dysplasia; Severe Myoclonic Epilepsy of Infancy (SMEI); Shaken Baby Syndrome; Shingles; Shy-Drager Syndrome; Sjogren's Syndrome; Sleep Apnea; Sleeping Sickness; Sotos Syndrome; Spasticity; Spina Bifida; Spinal Cord Infarction; Spinal Cord Injury; Spinal Cord Tumors; Spinal Muscular Atrophy; Spinocerebellar Atrophy; Spinocerebellar Degeneration; Steele-Richardson-Olszewski Syndrome; Stiff-Person Syndrome; Striatonigral Degeneration; Stroke; Sturge-Weber Syndrome; Subacute Sclerosing Panencephalitis; Subcortical Arteriosclerotic Encephalopathy; SUNCT Headache; Swallowing Disorders; Sydenham Chorea; Syncope; Syphilitic Spinal Sclerosis; Syringohydromyelia; Syringomyelia; Systemic Lupus Erythematosus; Tabes Dorsalis; Tardive Dyskinesia; Tarlov Cysts; Tay-Sachs Disease; Temporal Arteritis; Tethered Spinal Cord Syndrome; Thomsen's Myotonia; Thoracic Outlet Syndrome; Thyrotoxic Myopathy; Tic Douloureux; Todd's Paralysis; Tourette Syndrome; Transient Ischemic Attack; Transmissible Spongiform Encephalopathies; Transverse Myelitis; Traumatic Brain Injury; Tremor; Trigeminal Neuralgia; Tropical Spastic Paraparesis; Troyer Syndrome; Tuberous Sclerosis; Vascular Erectile Tumor; Vasculitis Syndromes of the Central and Peripheral Nervous Systems; Von Economo's Disease; Von Hippel-Lindau Disease (VHL); Von Recklinghausen's Disease; Wallenberg's Syndrome; Werdnig-Hoffman Disease; Wernicke-Korsakoff Syndrome; West Syndrome; Whiplash; Whipple's Disease; Williams Syndrome; Wilson Disease; Wolman's Disease; X-Linked Spinal and Bulbar Muscular Atrophy; Zellweger Syndrome, and/or the like.

In some embodiments, the supplements disclosed herein may also be administered to support the respiratory system of an equine, to support endothelial cell function. Administration of a compound in accordance with the present invention may spare the depletion of vitamin E and therefore improve the health of these horses or prevent and/or treat various physical and/or neurological disorders caused by vitamin E deficiencies. Many neurological disorders may be linked to genetic or exercise-induced vitamin E deficiencies, and treatment of such disorders by using a nutraceutical and/or supplement in accordance with the present disclosure is contemplated by and within embodiments of the present invention. In some embodiments, horses may have a genetic disposition such that their bodies can't store vitamin E. In cases where horses, or the like, have a genetic disposition in which their bodies can't store vitamin E, a supplement and/or nutraceutical in accordance with the present invention may be administered continuously and/or the like. In some embodiments, vitamin E deficiencies may produce muscle tremors, neurological head shaking, muscle spasms, and many more.

Embodiments of the present invention include mitigating or suppressing exercise-induced oxidative stress and its sequelae in race horses with the use of CoQ10 and vitamin E supplementation. In some embodiments “vitamin E” may comprise a complex comprising tocopherols and tocotrienols, or the like. After administration of a composition in accordance with the present disclosure, the risk for a number of neurological diseases may be reduced. In some embodiments, a composition may comprise from about 5 mg to about 20 g of broad spectrum tocotrienol and/or tocopherol vitamin E complex, for example, 1.5 mg tocotrienols. In some embodiments, a composition may comprise quercetin, curcumin, boswellia, Coenzyme Q10 (CoQ10), vitamin E, ribose, calcium carbonate, and/or zinc. Vitamin E may comprise a broad spectrum tocotrienol and/or tocopherol vitamin E complex.

A supplement, nutraceutical, and/or drug in accordance with embodiments of the present invention may help energize and maintain optimum mitochondrial functioning and peak-equine, human, cat, dog, and/or other mammal performance; may help support recovery of the horse after performance events; may help maintain healthy joints and muscles; help maintain a healthy inflammatory response in young race horses during training; and increase the bioavailability of vitamin E (e.g., a complex compressing tocopherols and tocotrienols), and/or the like. In some embodiments, a composition and/or supplement in accordance with embodiments of the present disclosure may be adapted to recycle and/or regenerate oxidized vitamin E, or the like. Many vitamin E supplements consist of natural or synthetic forms of alpha-tocopherol. As used herein, however, vitamin E, may comprise a complex nutrient comprising soluble naturally occurring compounds that form two groups; tocopherols (saturated) and tocotrienols (unsaturated). Each group may comprise four individual isoforms (α, β, γ and δ). A composition comprising quercetin, curcumin, boswellia, Coenzyme Q10 (CoQ10), vitamin E, ribose, calcium carbonate, and/or zinc, or the like may be comparatively gentle on the stomach when compared with other supplements. The quercetin may be provided in a base of rice syrup, thereby making the quercetin product high in amino acids.

Exercise-induced pulmonary hemorrhage (EIPH) is a serious problem in thoroughbred race horses or other horses that has been attributed to sustained strenuous exercise. EIPH is associated with poor performance in Thoroughbred race horses that are not medicated with diuretics and not using nasal dilator strips or other drugs. Exercise-induced oxidant stress as shown by increased production of reactive oxygen species (ROS) and free radicals may be present in horses and exercise-induced oxidant stress may be implicated in the pathogenesis of chronic inflammatory, vascular, and airway diseases in horses. Strenuous exercise may cause deleterious effects on pulmonary immune responses. High-intensity exhaustive exercise may lead to a chronic inflammatory state in horses that can affect their physical performance. Inflammation can increase the risk of developing EIPH. The risk of EIPH may be reduced by mitigating or suppressing exercise-induced oxidative stress and its sequelae in race horses by supplementation in accordance with the presentation, for example, supplementation with quercetin, curcumin, boswellia, CoQ10, and/or ribose.

In accordance with exemplary embodiments of the present disclosure, a composition including quercetin, curcumin, boswellia, CoQ10, and/or ribose may be administered to treat, prevent, and/or cure brain, heart, and vascular disorders and/or diseases in equine, feline, canine, humans, and other mammals. The elements of a composition in accordance with embodiments of the present invention may work synergistically to slow down the progression of inflammatory disorders and brain disorders such as dementia, Alzheimer's disease, and/or the like. The elements of a composition in accordance with embodiments of the present invention may also work synergistically to treat, prevent, and/or cure vascular diseases such as peripheral artery disease, aneurysm, renal artery disease, and/or the like.

Quercetin supplementation may significantly lower inflammatory markers after prolonged physical exertion, lower oxidative stress markers, improve performance in trained and untrained subjects, lower upper respiratory tract infection rates (like IAD), and lowering of C-reactive protein (CRP) (a key heart disease marker) in equine or human athletes after extreme exertion. Quercetin may be combined with rice solids that may comprise amino acids to help the healing building blocks in the body. The antioxidant and inflammatory response support may improve physical and mental performance by boosting energy, increasing endurance, and speeding recovery in both athletes and less active individuals. Quercetin increases the amount of mitochondria, which are the source of energy production in every cell in the body. Quercetin favorably alters markers of athletic performance as well as markers of exercise induced-inflammation.

A study was completed on the effects of quercetin on exercise potential and exercise-induced cytokines in horses. Six healthy, unfit Standardbred mares (˜500 kg, age 4-5 years) were assigned to one of two groups in a crossover fashion. The horses were dosed via a nasogastric tube BID (8 am and 8 PM) with 6 g of quercetin in tap water for 3.5 days. Two hours after the seventh and final dose, a graded exercise test (GXT) was run. During the test the treadmill remained at a fixed 6% grade. At T=−, the treadmill was started at a speed of 4 m/sec. Each following minute the treadmill speed was increased by 1 m/sec, up to a maximum speed of 11 m/sec. Blood and muscle samples were collected to measure markers of inflammation. There was a small but significant increase in the run time when the horses were dosed with quercetin, and recovery time was shortened significantly when compared to water-treated trials. There were exercise-induced increases in plasma TNF-α, IL-1, interferon-γ, granzyme-B (GrB), hematocrit, total protein, glucose, and lactate, but only with GrB was there a significant drug effect in the quercetin group. Intramuscular levels of IL-1 and GrB also increased significantly with exercise, but there was no effect from quercetin treatment. This study provided compelling evidence that quercetin could be useful in enhancing exercise performance, although the mechanism for this enhancement is unclear. While this study alone is not sufficient to recommend quercetin for mainstream usage, it adds to a growing body of literature supporting the drug's effectiveness.

Quercetin and/or other flavonoids (catechin, epigallocatechin-3-O-gallate, theaflavin, and/or the like may significantly reduce cellular inflammation and may generally outperform phenylbutazone by being effective at lower doses. In some embodiments a composition in accordance with the present disclosure may comprise curcumin. Curcumin may generally comprise a substance found in the spice turmeric and is generally not bioavaible. The bioavailable curcumin used in the formula contained herein, has powerful anti-inflammatory and antioxidant properties, helping reduce inflammatory markers that cause swelling and inflammation. Curcumin is a pleiotropic molecule that may be capable of interacting with molecular cellular markers involved in reducing inflammation. Curcumin may modulate the inflammatory response by down-regulating the activity of cyclooxygenase-2, lipoxygenase, and inducible nitric oxide synthase enzymes; and inhibits several other enzymes involved in inflammation mechanisms. Curcumin may also be used to treat, cure, and/or prevent various diseases, including equine, human, and mammal inflammatory airway disease (IAD), exercise-induced pulmonary hemorrhage (EIPH), emphysema, chronic obstructive pulmonary disease (COPD), multiple sclerosis (MS), respiratory diseases, multiple myeloma, pancreatic cancer, myelodysplastic syndromes, colon cancer, psoriasis, arthritis, major depressive disorder and Alzheimer's disease. When combined with quercetin bioavailability and absorption of curcumin may generally be increased due to the synergistic effect of combining these elements.

Compositions in accordance with embodiments of the present invention may prevent, treat, and/or cure these diseases. For example, compositions of the present invention may help cure or treat exercise-induced pulmonary hemorrhage (EIPH) for equine. Compositions in accordance with the present disclosure may also open the airway for more lung capacity for all professional athletes like runners, tennis players, eventing horses and any sport with excessive exertion. Compositions in accordance with the present invention may also be administered to humans to treat, cure, and/or prevent emphysema, chronic obstructive pulmonary disease (COPD), multiple sclerosis (MS), and/or other respiratory diseases.

In some embodiments, curcumin may comprise a complex of curcumin marketed under the name BCM-95®. In other embodiments, another type of curcumin may be used. BCM-95® fights joint-damaging effects of arthritis by attacking multiple inflammatory targets at once. BCM-95® may generally be more effective than arthritis drugs at treating arthritis while at a fraction of the cost. In addition, BCM-95® directly attacks inflammation, rather than simply masking pain and other symptoms like many drugs do. BCM-95® curcumin has a greater bioavailability than turmeric 95 percent extract and has 7 to 10 times better absorption than turmeric or plain curcumin. Curcumin is both a potent anti-inflammatory agent and a powerful stimulator for neurogenesis. Curcumin has a wide spectrum of activities including antioxidant and anti-inflammatory properties. ordinary

[symbol]

In accordance with exemplary embodiments of the present disclosure, a composition may also comprise boswellia. Boswellia may comprise anti-inflammatory properties and may be used to treat inflammatory conditions. Boswellia may also be included to treat inflammatory disorders and fever, rheumatism, gastrointestinal problems, and/or the like.

Compositions, nutraceuticals, supplements, and/or the like of the present invention may comprise CoQ10. In accordance with exemplary embodiments CoQ10 supplementation ameliorates inflammatory signaling and oxidative stress associated with strenuous exercise done with athletes, or the like. Supplementation with compositions in accordance with the present disclosure may help maintain healthy cytokines and inflammatory responses at the cellular levels, or the like.

As used herein, the term Coenzyme Q10 and its variants may generally refer to hydrosoluble Coenzyme Q10, or the like. Other ordinary types of Coenzyme Q10 may be used, but the dosage amounts required are significantly higher than hydrosoluble Coenzyme Q10, or the like. Some embodiments of the present disclosure may be used as a preventive and/or curative measure to treat any animal and/or humans with inflammation. For example, supplements, compositions, and/or nutraceuticals in accordance with the present invention may be administered to mares (when pregnant as a preventative measure, or the like), foals, yearlings, through aging equine, large and small animals and humans, and/or the like.

CoQ10, also known as ubiquinone, is a vitamin-like compound that has a fundamental role in cellular bioenergetics. CoQ10 is a cofactor in the mitochondrial electron transport chain (respiratory chain), and thus it has an obligatory role in the generation of biological energy in the form of adenosine triphosphate (“ATP”). CoQ10 supplementation may ameliorate inflammatory signaling and oxidative stress associated with strenuous exercise. CoQ10 may function as a mobile redox agent shuttling electrons and protons in the electron transport chain. Furthermore, CoQ10 in its reduced form as the hydroquinone (called “ubiquinol”) is a potent lipophilic antioxidant that is capable of recycling and regenerating other antioxidants and ascorbate (vitamin C). CoQ10 also has other important functions such as cell signaling and gene expression. CoQ10 is generally present in the blood serum of Thoroughbred race horses. In accordance with exemplary embodiments, providing dietary CoQ10 supplementation to horses results in a marked increase in serum CoQ10 concentrations and may prevent deterioration of the health of a horse after rigorous exercise.

In exemplary embodiments, the decrease in plasma CoQ10 following stress, exercise, for example breezes, may be attributed to severe oxidative stress brought on by more strenuous training. As such, in horses not receiving any CoQ10 supplementation, extreme stress or high intensity exercise may result in a marked depletion of plasma CoQ10 and this could impair the horse's health and physical performance.

In accordance with exemplary embodiments, a composition may comprise ribose. d-Ribose exerts anti-inflammatory effects and inhibits inflammation. It helps to improve athletic performance and the ability to exercise by boosting muscle energy. It has also been used to improve recovery and symptoms of chronic fatigue syndrome (CFS), fibromyalgia, and coronary artery disease. Ribose has been used to prevent symptoms such as cramping, pain, and stiffness after exercise in people with an inherited disorder called myoadenylate deaminase deficiency (MAD) or AMP deaminase deficiency (AMPD deficiency). Ribose has also been used to improve exercise ability in people with another inherited disorder called McArdle's disease.

In some embodiments, a composition may comprise quercetin in the amount of 2,000-15,000 mg. For example, the quercetin may be present in the amount of 2,000-10,000 mg. In some embodiments, a composition may comprise curcumin in the amount of 100-1,800 mg. For example, the curcumin may be present in the amount of 500 mg. In some embodiments, a composition may comprise boswellia in the amount of 100-1,800 mg. For example, the Boswellia may be present in the amount of 500 mg. In some embodiments hydrosoluble Coenzyme Q10 may be included in supplements and/or nutraceuticals in accordance with the present disclosure. In some embodiments, the Coenzyme Q10 may comprise and/or be included in the amount of 100 mg-3.4 g, or the like. For example, the CoQ10 may comprise and/or be included in the amount of 500 mg. In some embodiments, a composition may comprise Ribose in the amount of 2,000 mg-10,000 mg. For example, the Ribose may be present in the amount of 5,000 mg. In some embodiments, a composition may comprise Calcium Carbonate in the amount of 500 mg-2,500 mg. For example, the Calcium Carbonate may be present in the amount of 1,500 mg. Calcium Carbonate may be included as a buffer to help protect the digestive environment by buffering the stomach helping to prevent ulcers or irritation by curcumin, or the like.

In some embodiments, a composition may comprise zinc in the amount of 50-100 mg. For example, the zinc may be present in the amount of up to 100 mg. The combination of zinc, calcium carbonate, and/or curcumin help boost the immune system and buffer stomach acids, thus helping to prevent ulcers in the digestive tract of monogastric organisms. In exemplary embodiments, using a supplement comprising quercetin, curcumin, boswellia, CoQ10, ribose, calcium carbonate, and/or zinc would protect a monogastric organisms stomach while also helping maintain healthy joints and muscles, maintain healthy inflammatory responses, and/or the like.

Numerous studies have documented alterations in pro-inflammatory cytokine expression in response to exercise in humans and large and small animals. Equine athletes likewise exhibit positive inflammatory responses to pre & post exercise. High volume/intensity training can produce muscle, skeletal or joint trauma. Free radical generation can be estimated by the measurement of products of lipid peroxidation such as MDA whose level is increased in equine blood and tissues during exercise. The accumulation of lactic acid is a functional measure of anaerobic metabolism associated with high intensity exercise. Together, these multiple insults activate circulating monocytes and other cells to produce pro-inflammatory cytokines. These changes in pro-inflammatory gene expression occur throughout the body, especially in skeletal muscle and peripheral blood cells. Peripherally produced cytokines can in turn augment or exacerbate localized inflammatory responses in joints and muscles thus leading to an amplification of the inflammatory cascade. A similar anti-inflammatory effect was found due to exercise training on pro-inflammatory cytokine gene expression in peripheral blood cells is present in young Thoroughbreds when exercising.

The initial effect of exercise bouts may be the intensity-dependent accumulation of lactic acid. Lactate accumulation may generally be detected within 5 minutes of the completion of exercise. The levels of lactate in the blood samples are generally proportional to the effort expended in exercise, with maximal accumulation occurring in response to the higher speed tests. An increase in MDA may also be present post-exercise.

Concurrent with the rise in lactate and MDA, a significant increase in two indirect markers for LAK activity, granzyme B and IFN-γ, may occur immediately post exercise. This increase in LAK function is likely the result of repartitioning of the cells in response to exercise and the increased cytotoxic activity of the cells in response to increased cytokine or other mediator production. The overall effect may be intensity dependent as there may be no effect of the initial light exercise test on either marker, similar to what may occur to LAK function following low intensity exercise. There may be no effect of exercise training on pre-exercise or baseline IFN-γ and granzyme B expression throughout a training period.

Two hours after completing exercise tests horses may exhibit increased expression of Inflammatory Markers IL-1β mRNA in their peripheral blood. The expression of IL-1β may be exercise-intensity-dependent. The temporal association between cytokine production and markers of oxidative inflammatory damage may demonstrate that damage to internal organs and muscles is caused by the production of inflammatory markers, such as, cytokines. Baseline expression of TNFα may decrease with exercise and may be associated with an overall trend for IL-1β baseline expression to also decrease. This overall decline in pro-inflammatory cytokine expression may significant for those horses receiving a supplement in accordance with embodiments of the present disclosure.

The adaptation of using the nutraceuticals in accordance with embodiments of the present disclosure when exercising, may lead to reduced inflammatory responses in trained human and equine athletes. After a supplement and/or composition in accordance with the present disclosure, a monogastric organism may experience reduction in pro-inflammatory gene expression after exercise. This may be evidenced by both an overall decline in Inflammatory Markers

A Nutraceutical in accordance with embodiments of the present disclosure may comprise quercetin, ribose, calcium carbonate, hydrosoluble Coenzyme Q10 (CoQ10), curcumin, boswellia, and zinc, or the like, which may increase exercise capacity in humans, equine, small animals and monogastric organisms, or the like, by helping maintain healthy Inflammatory levels in the treated horses and monogastric organisms.

In accordance with exemplary embodiments of the present invention, quercetin, curcumin, boswellia, CoQ10, ribose, calcium carbonate, and zinc, may be administered to horses via a horse feed mixture and/or a syringe for improving the health of work and performance horses, including thoroughbred race horses. Hydrosoluble quercetin, ribose, calcium carbonate, hydrosoluble Coenzyme Q10 (CoQ10), hydrosoluble curcumin, boswellia, and zinc, supplementation in accordance with embodiments of the present invention may lead to improvements in health and performance, and prevention and/or treatment of many physical and/or inflammatory disorders. In some embodiments, compositions, supplements, and/or nutraceuticals in accordance with the present invention may be used to prevent and/or treat horses, humans, or other animals for conditions, including physical disorders, which may be naturally occurring or may occur as a result of stress, exercise, and/or the like. There is a marked increase in inflammatory markers when horses are subjected to stress or exercise such as high intensity training, and this could possibly lead to impaired health Inflammatoy disorders, thus resulting in poor physical performance.

A supplement in accordance with exemplary embodiments may be administered to horses of varying ages, or the like. For example a supplement or a preventative nutraceutical in accordance with exemplary embodiments of the present invention may be administered to one to two-year old and older, performance and work horses or it may be administered to horses with inflammation to help bring the body back to its natural inflammatory state. A supplement in accordance with exemplary embodiments may be administered by oral dietary supplementation, of the like. For example, a supplement may be administered with feed for horses and/or the like. The supplement may also be administered via a syringe, or the like.

In exemplary embodiments, horses that may be administered quercetin, curcumin, boswellia, CoQ10, ribose, calcium carbonate, and zinc, for health improvement may include horses in various weights. By way of example, the weight of the horses may range from about 100 kg to about 570 kg with a mean of 545 kg. In some embodiments, the dosage of quercetin, curcumin, boswellia, CoQ10, ribose, calcium carbonate, and zinc, may be administered in different doses calculated on a body weight basis. A 220 lb horse may be administered (a) 20-30% of the dose of a 1200 lb horse.

In some embodiments, the supplementation may be continued indefinitely, or for a specific period of time. For example, supplementation may continue during a time period in which a horse is regularly engaged in exercise activities. In one embodiment, quercetin, curcumin, boswellia, CoQ10, ribose, calcium carbonate, and zinc may be administered two times daily for 30 days, then one time daily for 60, 90 days or a total of 120 days or, ongoing as a preventative, during which periods the horses may be performing exercising daily, galloping and breezing. The results will begin to show reduced inflammatory markers in the blood almost immediately and the user should discern improvement in within 2-4 weeks. The supplement should continue to be administered even after the inflammatory levels return to normal to help maintain and stabilize the healthy inflammatory levels. Shown first in drawn bloods, then in the gradual decrease in inflammatory symptoms within the area(s) showing sign(s) of inflammation. The results will be ongoing and with healthy inflammatory responses progressing from within the body.

In some embodiments, an exercise regimen may be implemented for the horse by the administrative user of the invention. In some embodiments, an exercise regimen may comprise one or more bouts or sessions of physical exercise, such as work like pulling a wagon, working in a field, dressage, eventing, jumping, or graduated training, low-intensity exercise (also called “galloping”), high-intensity exercise (also called “breezing”), and/or the like. In particular non-limiting embodiments, the physical exercise may be a one-time session, but in other non-limiting embodiments, the sessions of physical exercise may be repeated over time, and in other embodiments the repeated-over-time sessions may show variations of type and intensity throughout the repetitions. During these sessions of work or training, harmful inflammatory cytokines are beginning to build up within the body. When the body can no longer control the harmful cytokines and the body becomes overwhelmed injuries occur. Thus, before or after any exercise regimen is completed, as a preventative, the nutritional supplements should be administered as a shield to the horse, small animal or Human, to help the body prevent injuries and remain in a healthy inflammatory state. If the body is overwhelmed and an inflammatory injury occurs, then the nutraceuticals embodied here within, will help the body bring itself back to a healthy inflammatory state. Thus inflammatory injury will be brought under the body's control.

In some embodiments, an additional comparison may be made between Inflammatory health states of a horse using methods described herein. The compared Inflammatory health states may comprise states before implementation of an exercise regimen and states after implementation thereof. Some embodiments of the invention may loop through the various steps and combinations and sequences thereof. Upon achieving a satisfactory result (e.g., by preventing injury or helping to maintain a desired health state of the horse, or a desired result of comparison between health states of the horse), the method may terminate instead of iterating back to an initial step in accordance with the present disclosure. It should be understood that the order, sequence, and timing of any of the steps identified herein are for illustrative purposes only and should not to be read as limiting. Variations of such steps as to order, sequence, and timing, are to be considered as included herein and incorporated hereby into the present disclosure. The nutraceuticals mentioned herein are for use as a preventative or to help bring the body back to healthy state by helping the body maintain healthy inflammatory levels.

Quercetin may be provided in hydrosoluble form, or the like, as a polar auxin transport inhibitor and to increase the efficacy of other elements. Quercetin may comprise, in some embodiments, QU995, which may comprise a 99.5% or greater purity of quercetin, or the like. In some embodiments, bioavailable curcumin & CoQ10 may comprise a highly bioavailable form of aqueous hydrosoluble CoQ10 and curcumin, or the like. A supplement, nutraceutical, and/or drug in accordance with embodiments of the present invention may help maintain healthy inflammatory levels by acting as an inflammatory shield, and help shield the body against inflammatory injuries. Research conducted by the inventors has demonstrated that this unique combination of nutraceuticals has significantly improved inflammatory markers throughout the body. Thus showing a positive effect in the muscles, heart, and organs through the equine, human, and small animal bodies, while helping to maintain optimum mitochondrial functioning and peak performance in all athletes, including horses, humans, and/or the like. This synergistic combination of helps support recovery of work animals, of Performance Athletes i.e.; human, horse before or after performance events; may help maintain healthy joints and muscles; help maintain healthy cardio and vascular functioning, and help maintain a healthy inflammatory response in young race horses during training. By combining the ingredients, the effects of each ingredient may be doubled when compared to the effectiveness of each ingredient administered separately.

A composition in accordance with exemplary embodiments of the present disclosure may also comprise curcumin. Curcumin can sometimes irritate the stomach and contribute to stomach ulcers. Curcumin may irritate the stomach of the user following administration. Curcumin may generally be harsh on the stomach and often cause ulcers, upset stomach (i.e., gastroenteritis), or other physical conditions in monogastric organisms. To prevent and/or treat these conditions, Zn as a healing mineral and/or calcium carbonate as a buffering mineral may also be included, thereby making the composition easier on the stomach, or the like.

A composition in accordance with exemplary embodiments of the present disclosure may also comprise zinc. Zinc may be included for curative benefits for stomach irritations and/or to prevent irritations that may be caused by administration of curcumin. Zinc complexes may have anti-ulcer effects as it acts as an anti-inflammatory and antioxidant. When combined with curcumin, calcium carbonate, and Zn these minerals will promote a buffering of the stomach acids & healing of gastric ulcers and/or wounds and thus be gentle on the stomach. Zn and curcumin may also comprise antioxidant properties.

In accordance with exemplary embodiments, a composition may also comprise calcium carbonate as a buffering agent to help prevent stomach upset & gastric ulcers, or the like. Calcium carbonate may be used as an antacid, or a substance that neutralizes stomach acidity. Calcium carbonate may be included in a composition in accordance with exemplary embodiments to prevent stomach irritation, gastroenteritis, or the like.

The epithelial layer may be a first point of contact for inhaled substances. In both humans and horses, epithelial cells lining the respiratory tract form an interface between external and internal environments. The airway epithelium actively participates in lung defense and inflammatory responses. Epithelial cells contain receptors that engage cell wall components of infectious organisms, enabling viruses, bacteria and fungi to gain entrance into the epithelial cells and cause disease. Compositions in accordance with embodiments of the present invention may prevent, treat, and/or cure these diseases. For example, compositions of the present invention may help prevent, cure, or treat exercise-induced pulmonary hemorrhage (EIPH) and (IAD) for equine, humans, performance athletes, small animals, and/or other mammals. Compositions in accordance with the present disclosure may also open the airway for more lung capacity for all professional athletes like runners, tennis players, eventing horses and any sport with excessive exertion. Compositions in accordance with the present invention may also be administered to humans to treat, cure, and/or prevent emphysema, chronic obstructive pulmonary disease (COPD), multiple sclerosis (MS), and/or other respiratory diseases.

While the foregoing is directed to embodiments of the present invention, other and further embodiments of the invention may be devised without departing from the basic scope thereof. It is also understood that various embodiments described herein may be utilized in combination with any other embodiment described, without departing from the scope contained herein.

What is claimed is:

1. An anti-inflammatory composition for monogastric organisms, the composition comprising:
quercetin;
curcumin;
boswellia;
Coenzyme Q10 (CoQ10); and
ribose.

2. The composition of claim 1, further comprising:
calcium carbonate; and
zinc.

3. The composition of claim 2, wherein the calcium carbonate is present in the amount of 1,500 mg and the zinc is present in the amount of 50-100 mg.

4. The composition of claim 1, further comprising vitamin E.

5. The composition of claim 4, wherein the vitamin E comprises a complex comprising tocopherols and tocotrienols.

6. The composition of claim 1, wherein:
the quercetin is present in the amount of 2,000-10,000 mg;
the curcumin is present in the amount of 250-500 mg;
the boswellia is present in the amount of 250-500 mg;
the CoQ10 is present in the amount of 200-1,000 mg; and
the ribose is present in the amount of 5,000 mg.

7. The composition of claim 1, further comprising vitamin C, magnesium, and selenium.

8. The composition of claim 1, further comprising at least one selected from the group of: pyrroloquinoline quinone, Omega 3, magnesium, folic Acid, and selenium.

9. A method of counteracting exercise-induced inflammation in a horse, the method comprising:
administering an effective amount of quercetin, curcumin, boswellia, Coenzyme Q10 (CoQ10), ribose, calcium carbonate, and zinc, thereby maintaining a healthy inflammatory response during exercise.

10. The composition of claim 9, wherein:
the quercetin is present in the amount of 2,000-10,000 mg;
the curcumin is present in the amount of 250-500 mg;
the boswellia is present in the amount of 250-500 mg;
the CoQ10 is present in the amount of 200-1,000 mg;
the ribose is present in the amount of 5,000 mg;
the calcium carbonate is present in the amount of 1,500 mg-2500 mg; and
the zinc is present in the amount of 50-100 mg.

11. The composition of claim 9, further comprising vitamin C, magnesium, and selenium.

12. The composition of claim 9, further comprising at least one selected from the group of: pyrroloquinoline quinone, Omega 3, magnesium, folic Acid, and selenium.

13. A method for ascertaining and comparing results of using nutritional supplements to counteract exercise-induced causing harmful levels of inflammation and its sequelae in a horse, the method comprising:
determining dietary supplement amounts of quercetin, curcumin, boswellia, Coenzyme Q10 (CoQ10), ribose, calcium carbonate, and zinc, wherein the dietary supplement amounts are based at least in part on the characteristics of the horse;
administering to the horse a dietary supplement comprising quercetin, curcumin, boswellia, CoQ10, ribose, calcium carbonate, and zinc, and zinc in the determined dietary supplement amounts; and
ascertaining a health state of the horse, the ascertained health state of the horse being indicative of exercise-induced inflammation and its sequelae in the horse.

14. The method of claim 13 further comprising:
modifying the determined supplement amounts based at least in part on the ascertained health state of the horse as preventative or curative;
administering to the horse a dietary supplement comprising quercetin, curcumin, boswellia, CoQ10, ribose, calcium carbonate, and zinc, in the modified dietary supplement amounts;
ascertaining with an equine blood-analysis apparatus, a health state of the horse, the ascertained health state of the horse being indicative of exercise-induced oxidative stress and inflammation and its sequelae in the horse; and
determining a result, thereby ascertaining a comparison between the ascertained health states of the horse, the determined result being indicative of using quercetin, curcumin, boswellia, CoQ10, ribose, calcium carbonate, and zinc as dietary supplements to counteract exercise-induced inflammation and its sequelae in a horse.

15. The method of claim 14 further comprising:
implementing an exercise regimen for the horse, the exercise regimen comprising one or more sessions of physical activity, the physical activity comprising one or more of graduated training, low-intensity exercise or galloping, and high-intensity exercise or breezing; and
modifying the determined supplement amounts based at least in part on the implemented exercise regimen for the horse;
administering to the horse a dietary supplement comprising quercetin, curcumin, boswellia, CoQ10, ribose, calcium carbonate, and zinc in the modified dietary supplement amounts;
ascertaining, with the equine blood-analysis apparatus, a health state of the horse, the ascertained health state of the horse being indicative of exercise-induced oxidative stress and its sequelae in the horse; and
determining a result of a comparison between the ascertained health states of the horse, the compared ascertained health states being indicative of the impact of using quercetin, curcumin, boswellia, CoQ10, ribose, calcium carbonate, and zinc as dietary supplements on exercise-induced oxidative stress and its sequelae in a horse.

16. The method of claim 15, wherein the vital characteristics of the horse comprise one or more of: age, weight, height, gender, breed, size, racing category, performance category, labor category, and farming category of the horse or status as being a racing Thoroughbred or performance horse.

17. The method of claim 16, wherein the periodic basis comprises at least one of: hourly, daily, twice weekly, weekly, twice monthly, monthly, twice annually, and annually.

18. The composition of claim 13, wherein:
the quercetin is present in the amount of 2,000-10,000 mg;
the curcumin is present in the amount of 250-500 mg;
the boswellia is present in the amount of 250-500 mg;
the CoQ10 is present in the amount of 200-1,000 mg;
the ribose is present in the amount of 5,000 mg;
the calcium carbonate is present in the amount of 1,500 mg-2500 mg; and
the zinc is present in the amount of 50-100 mg.

19. The composition of claim 13, further comprising vitamin C, magnesium, and selenium.

20. The composition of claim 13, further comprising at least one selected from the group of: pyrroloquinoline quinone, Omega 3, magnesium, folic Acid, and selenium.
